(19) United States
(12) Patent Application Publication
(10) Pub. No.: US 2026/0027172 A1
(43) Pub. Date: Jan. 29, 2026

(54) LIVER-PROTECTING MILK THISTLE FORMULA COMPOSITION, AND PREPARATION METHOD THEREOF

(71) Applicant: NANO BIOLOGY LIMITED, RENTON, WA (US)

(72) Inventors: Xinxin LU, Ankang (CN); Likang CHEN, Ankang (CN); Yukang ZHANG, Ankang (CN)

(73) Assignee: NANO BIOLOGY LIMITED, RENTON, WA (US)

(21) Appl. No.: 18/782,075

(22) Filed: Jul. 24, 2024

(51) Int. Cl. A61K 36/28; A61K 9/48; A61K 31/197; A61K 31/352; A61K 31/357; A61K 36/288; A61K 38/06

(52) U.S. Cl. CPC A61K 36/28; A61K 9/4833; A61K 31/197; A61K 31/352; A61K 31/357; A61K 36/288; A61K 38/063; A61K 2236/15

(57) ABSTRACT

The present invention relates to the technical field of liver-protecting formula compositions, and in particular, to a liver-protecting milk thistle formula composition, and a preparation method thereof. Technical problems: the liver-protecting milk thistle formula composition, and a preparation method thereof are intended to solve the technical problems that most of existing liver-protecting compositions in the prior art are designed for the general liver protection needs of the general population and cannot specifically treat liver damage caused by excessive alcohol intake and liver diseases such as cirrhosis, acute hepatitis, hepatitis, fatty liver, cholangitis, cholelithiasis, psoriasis, and hypercholesterolemia. Technical solution: a liver-protecting milk thistle formula composition, including the following components: silymarin, puerarin, artichoke extract, glutathione, dandelion extract, and GABA. The liver-protecting formula composition has a good therapeutic effect on liver damage caused by excessive alcohol intake, and liver diseases such as cirrhosis, acute hepatitis, hepatitis, fatty liver, cholangitis, cholelithiasis, psoriasis, and hypercholesterolemia.

### TECHNICAL FIELD

The present invention relates to the technical field of liver-protecting formula compositions, and in particular, to a liver-protecting milk thistle formula composition, and a preparation method thereof.

### BACKGROUND

Existing liver-protecting compositions on the market have relatively simple functions, and most of them are designed for the general liver protection needs of the general population and can neither be customized according to individual circumstances and needs nor specifically treat liver damage caused by excessive alcohol intake and liver diseases such as cirrhosis, acute hepatitis, hepatitis, fatty liver, cholangitis, cholelithiasis, psoriasis, and hypercholesterolemia. In Europe, milk thistle has been used to treat liver diseases for thousands of years. Milk thistle can activate liver protein regeneration, restore activity of liver cells damaged by alcohol and viruses, scavenge free radicals, maintain the permeability of liver cell membranes, reduce liver damage caused by toxins, protect people who suffer from liver damage due to long-term drinking or have acute or chronic hepatitis, fatty liver, cirrhosis, or toxic liver damage. The liver-protecting formula composition provided by the present invention has a good therapeutic effect on liver damage caused by excessive alcohol intake, and liver diseases such as cirrhosis, acute hepatitis, hepatitis, fatty liver, cholangitis, cholelithiasis, psoriasis, and hypercholesterolemia.

### SUMMARY

In view of the shortcomings of the prior art, the present invention is intended to provide a liver-protecting milk thistle formula composition, and a preparation method thereof to solve the technical problems that existing liver-protecting compositions in the prior art have relatively simple functions, and most of them are designed for the general liver protection needs of the general population and cannot specifically treat liver damage caused by excessive alcohol intake and liver diseases such as cirrhosis, acute hepatitis, hepatitis, fatty liver, cholangitis, cholelithiasis, psoriasis, and hypercholesterolemia.

A technical solution of the present invention is as follows: a liver-protecting milk thistle formula composition, including the following components: 50-500 mg of silymarin, 30-300 mg of puerarin, 10-100 mg of artichoke extract, 10-100 mg of glutathione, 10-100 mg of dandelion extract, and 10-200 mg of GABA.

Preferably, the mass ratio of the puerarin, the glutathione, and the dandelion extract is 3:1:1.

A preparation method of a liver-protecting milk thistle formula composition is further provided, where the liver-protecting milk thistle formula composition includes the above-described one, and the method includes the following steps:
S1: grinding of raw materials
   use a grinder to grind raw materials such as milk thistle, Pueraria lobata,
   artichokes, glutathione, and dandelions into powder after drying them according to a formula;
S2: extraction and concentration
   use an ultrasonic extractor or a thermal reflux extractor to extract active ingredients from the raw materials ground in the step S1, filter the extracts to remove residues, and then concentrate the extracts to obtain concentrated extracts;
S3: mixing
   accurately weigh 50-500 mg of concentrated silymarin, 30-300 mg of puerarin, 10-100 mg of artichoke extract, 10-100 mg of glutathione, 10-100 mg of dandelion extract, and 10-200 mg of GABA, and add the ingredients into a mixing mortar and mix them thoroughly to ensure each of the ingredients is evenly distributed;
S4: packaging
   use a capsule filling machine to fill the mixed powder into capsules, and then use a packaging machine to package the capsules; and
S5: quality inspection
   use a quality control instrument to conduct quality inspection on a final product, including ingredient content, uniformity, stability, etc., to ensure that the product meets prescribed standards.

Preferably, the raw materials of Chinese herbal medicine in the step S1 can be dried in an oven, and the milk thistle and the Pueraria lobata are dried at 50-60° C. for 2-4 hours; the artichokes and the dandelions are dried at 40-50° C. for 3-5 hours; and the glutathione is dried at 60-70° C. for 1-3 hours.

Preferably, the grinder in the step S1 can be a blade grinder with a rotation speed of 5,000 to 10,000 rpm.

Preferably, a solvent used in the extraction in the step S2 is methanol, ethanol, or water.

Preferably, the mixing in the step S3 is carried out at a speed of 60 to 120 rpm for 10 to 20 minutes.

Preferably, in the step S5, a high-performance liquid chromatograph can be used to analyze content and purity of the ingredients in the formula composition, and a surface tensiometer can be used to evaluate surface properties of the formula composition, etc.

The present invention has the following beneficial effects: in the liver-protecting formula composition provided by the present invention, the silymarin and the dandelion extract can protect liver health, promote the repair and regeneration of liver cells, reduce liver damage, help scavenge free radicals and toxic substances, and relieve oxidative stress and inflammatory response in the liver, the silymarin, the artichoke extract, and the dandelion extract all contain compounds with anti-inflammatory and antioxidant activities to relieve inflammatory response and oxidative stress damage and protect cell health, the puerarin has anti-ulcer and anti-inflammatory effects to improve gastrointestinal health and relieve inflammatory response, the silymarin and the puerarin also have the effect of lowering cholesterol and blood lipids to promote cardiovascular health, and the GABA is a neurotransmitter that has calming and relaxing effects to improve sleep quality and reduce stress; therefore, the liver-protecting formula composition provided by the present invention has a good therapeutic effect on liver damage caused by excessive alcohol intake, and liver diseases such as cirrhosis, acute hepatitis, hepatitis, fatty liver, cholangitis, cholelithiasis, psoriasis, and hypercholesterolemia.

### DETAILED DESCRIPTION OF THE EMBODIMENTS

### Embodiment 1

The present invention will be further described in conjunction with embodiments.

The present invention provides an embodiment as follows: a liver-protecting milk thistle formula composition, including the following components: 50 mg of silymarin, 30 mg of puerarin, 10 mg of artichoke extract, 10 mg of glutathione, 10 mg of dandelion extract, and 10 mg of GABA, where the mass ratio of the puerarin, the glutathione, and the dandelion extract is 3:1:1.

A preparation method of a liver-protecting milk thistle formula composition is further provided, where the liver-protecting milk thistle formula composition includes the above-described one, and the method includes the following steps:
S1: grinding of raw materials
   use a grinder to grind raw materials such as milk thistle, Pueraria lobata, artichokes, glutathione, and dandelions into powder after drying them according to a formula, where the raw materials can be dried in an oven, the milk thistle and the Pueraria lobata are dried at 50° C. for 2 hours; the artichokes and the dandelions are dried at 40° C. for 3 hours, the glutathione is dried at 60° C. for 1 hour, and the grinder can be a blade grinder with a rotation speed of 5,000 to 10,000 rpm;
S2: extraction and concentration
   use an ultrasonic extractor or a thermal reflux extractor to extract active ingredients from the raw materials ground in the step S1, filter the extracts to remove residues, and then concentrate the extracts to obtain concentrated extracts, where a solvent used in the extraction is methanol, ethanol, or water;
S3: mixing
   accurately weigh 50 mg of concentrated silymarin, 30 mg of puerarin, 10 mg of artichoke extract, 10 mg of glutathione, 10 mg of dandelion extract, and 10 mg of GABA, and add the ingredients into a mixing mortar and mix them thoroughly at a speed of 60 rpm for 10 minutes to ensure each of the ingredients is evenly distributed;
S4: packaging
   use a capsule filling machine to fill the mixed powder into capsules, and then use a packaging machine to package the capsules; and
S5: quality inspection
   use a quality control instrument to conduct quality inspection on a final product, including ingredient content, uniformity, stability, etc., to ensure that the product meets prescribed standards, where a high-performance liquid chromatograph can be used to analyze content and purity of the ingredients in the formula composition, and a surface tensiometer can be used to evaluate surface properties of the formula composition, etc.

### Embodiment 2

The present invention will be further described in conjunction with embodiments.

The present invention provides an embodiment as follows: a liver-protecting milk thistle formula composition, including the following components: 300 mg of silymarin, 240 mg of puerarin, 70 mg of artichoke extract, 80 mg of glutathione, 80 mg of dandelion extract, and 140 mg of GABA, where the mass ratio of the puerarin, the glutathione, and the dandelion extract is 3:1:1.

A preparation method of a liver-protecting milk thistle formula composition is further provided, where the liver-protecting milk thistle formula composition includes the above-described one, and the method includes the following steps:
S1: grinding of raw materials
   use a grinder to grind raw materials such as milk thistle, Pueraria lobata, artichokes, glutathione, and dandelions into powder after drying them according to a formula, where the raw materials can be dried in an oven, the milk thistle and the Pueraria lobata are dried at 55° C. for 3 hours; the artichokes and the dandelions are dried at 45° C. for 4 hours, the glutathione is dried at 65° C. for 2 hours, and the grinder can be a blade grinder with a rotation speed of 5,000 to 10,000 rpm;
S2: extraction and concentration
   use an ultrasonic extractor or a thermal reflux extractor to extract active ingredients from the raw materials ground in the step S1, filter the extracts to remove residues, and then concentrate the extracts to obtain concentrated extracts, where a solvent used in the extraction is methanol, ethanol, or water;
S3: mixing
   accurately weigh 300 mg of concentrated silymarin, 240 mg of puerarin, 70 mg of artichoke extract, 80 mg of glutathione, 80 mg of dandelion extract, and 140 mg of GABA, and add the ingredients into a mixing mortar and mix them thoroughly at a speed of 90 rpm for 15 minutes to ensure each of the ingredients is evenly distributed;
S4: packaging
   use a capsule filling machine to fill the mixed powder into capsules, and then use a packaging machine to package the capsules; and
S5: quality inspection
   use a quality control instrument to conduct quality inspection on a final product, including ingredient content, uniformity, stability, etc., to ensure that the product meets prescribed standards, where a high-performance liquid chromatograph can be used to analyze content and purity of the ingredients in the formula composition, and a surface tensiometer can be used to evaluate surface properties of the formula composition, etc.

### Embodiment 3

The present invention will be further described in conjunction with embodiments.

The present invention provides an embodiment as follows: a liver-protecting milk thistle formula composition, including the following components: 500 mg of silymarin, 300 mg of puerarin, 100 mg of artichoke extract, 100 mg of glutathione, 100 mg of dandelion extract, and 200 mg of GABA, where the mass ratio of the puerarin, the glutathione, and the dandelion extract is 3:1:1.

A preparation method of a liver-protecting milk thistle formula composition is further provided, where the liver-protecting milk thistle formula composition includes the above-described one, and the method includes the following steps:
S1: grinding of raw materials
   use a grinder to grind raw materials such as milk thistle, Pueraria lobata, artichokes, glutathione, and dandelions into powder after drying them according to a formula, where the raw materials can be dried in an oven, the milk thistle and the Pueraria lobata are dried at 60° C. for 4 hours; the artichokes and the dandelions are dried at 50° C. for 5 hours, the glutathione is dried at 70° C. for 3 hours, and the grinder can be a blade grinder with a rotation speed of 5,000 to 10,000 rpm;
S2: extraction and concentration
   use an ultrasonic extractor or a thermal reflux extractor to extract active ingredients from the raw materials ground in the step S1, filter the extracts to remove residues, and then concentrate the extracts to obtain concentrated extracts, where a solvent used in the extraction is methanol, ethanol, or water;
S3: mixing
   accurately weigh 500 mg of concentrated silymarin, 300 mg of puerarin, 100 mg of artichoke extract, 100 mg of glutathione, 100 mg of dandelion extract, and 200 mg of GABA, and add the ingredients into a mixing mortar and mix them thoroughly at a speed of 120 rpm for 20 minutes to ensure each of the ingredients is evenly distributed;
S4: packaging
   use a capsule filling machine to fill the mixed powder into capsules, and then use a packaging machine to package the capsules; and
S5: quality inspection
   use a quality control instrument to conduct quality inspection on a final product, including ingredient content, uniformity, stability, etc., to ensure that the product meets prescribed standards, where a high-performance liquid chromatograph can be used to analyze content and purity of the ingredients in the formula composition, and a surface tensiometer can be used to evaluate surface properties of the formula composition, etc.

### Experimental Example

100 appropriate experimental rats were selected and divided into multiple groups, including a blank control group of 20 rats, a control group of 20 rats receiving a drug known to have a liver protection effect, and three experimental groups of 20 rats each, with each of the experimental groups receiving the liver-protecting milk thistle formula at a different concentration. The experimental rats were fed or injected with corresponding drugs at regular intervals, and an equal amount of solvent was administered to the control group. After one month of administration, the experimental rats were dissected or their blood samples were taken to detect and compare liver function indicators in different groups, such as serum alanine transaminase (ALT), serum aspartate aminotransferase (AST), and total bilirubin (TBIL). The experimental data are as follows:

| | Embodiment | | | Drug | Blank |
|---|---|---|---|---|---|
| | Embodi- | Embodi- | Embodi- | Control | Control |
| Item | ment 1 | ment 2 | ment 3 | Group | Group |
| ALT (U/L) | 31 | 36 | 40 | 45 | 60 |
| AST (U/L) | 29 | 33 | 37 | 42 | 65 |
| TBIL (mg/dL) | 0.6 | 1 | 0.8 | 1.3 | 2 |

What is claimed is:

1. A liver-protecting milk thistle formula composition, comprising the following components: 50-500 mg of silymarin, 30-300 mg of puerarin, 10-100 mg of artichoke extract, 10-100 mg of glutathione, 10-100 mg of dandelion extract, and 10-200 mg of GABA.

2. The liver-protecting milk thistle formula composition of claim 1, wherein the mass ratio of the puerarin, the glutathione, and the dandelion extract is 3:1:1.

3. A preparation method of a liver-protecting milk thistle formula composition, wherein the liver-protecting milk thistle formula composition comprises the one according to claim 1, and the method comprises the following steps:
S1: grinding of raw materials
use a grinder to grind raw materials such as milk thistle, Pueraria lobata, artichokes, glutathione, and dandelions into powder after drying them according to a formula;
S2: extraction and concentration
use an ultrasonic extractor or a thermal reflux extractor to extract active ingredients from the raw materials ground in the step S1, filter the extracts to remove residues, and then concentrate the extracts to obtain concentrated extracts;
S3: mixing
accurately weigh 50-500 mg of concentrated silymarin, 30-300 mg of puerarin, 10-100 mg of artichoke extract, 10-100 mg of glutathione, 10-100 mg of dandelion extract, and 10-200 mg of GABA, and add the ingredients into a mixing mortar and mix them thoroughly to ensure each of the ingredients is evenly distributed;
S4: packaging
use a capsule filling machine to fill the mixed powder into capsules, and then use a packaging machine to package the capsules; and
S5: quality inspection
use a quality control instrument to conduct quality inspection on a final product, including ingredient content, uniformity, stability, etc., to ensure that the product meets prescribed standards.

4. The preparation method of a liver-protecting milk thistle formula composition according to claim 3, wherein the raw materials of Chinese herbal medicine in the step S1 can be dried in an oven, and the milk thistle and the Pueraria lobata are dried at 50-60° C. for 2-4 hours; the artichokes and the dandelions are dried at 40-50° C. for 3-5 hours; and the glutathione is dried at 60-70° C. for 1-3 hours.

5. The preparation method of a liver-protecting milk thistle formula composition according to claim 3, wherein the grinder in the step S1 can be a blade grinder with a rotation speed of 5,000 to 10,000 rpm.

6. The preparation method of a liver-protecting milk thistle formula composition according to claim 3, wherein a solvent used in the extraction in the step S2 is methanol, ethanol, or water.

7. The preparation method of a liver-protecting milk thistle formula composition according to claim 3, wherein the mixing in the step S3 is carried out at a speed of 60 to 120 rpm for 10 to 20 minutes.

8. The preparation method of a liver-protecting milk thistle formula composition according to claim 3, wherein in the step S5, a high-performance liquid chromatograph can be used to analyze content and purity of the ingredients in the formula composition, and a surface tensiometer can be used to evaluate surface properties of the formula composition, etc.
